(19) United States
(12) Patent Application Publication
(10) Pub. No.: US 2016/0244791 A1
(43) Pub. Date: Aug. 25, 2016

(54) METHODS AND ENZYMES FOR PRODUCING HYDROXYMETHYLFURFURAL

(71) Applicant: THE UNIVERSITY OF FLORIDA RESEARCH FOUNDATION, INC., Galnesville, FL (US)

(72) Inventors: NICOLE A. HORENSTEIN, GAINESVILLE, FL (US); STEVEN DOUGLAS BRUNER, GAINESVILLE, FL (US); ADRIAN ROITBERG, GAINESVILLE, FL (US); NICOLAS C. POLFER, GAINESVILLE, FL (US)

(21) Appl. No.: 15/025,022

(22) PCT Filed: Sep. 26, 2014
(86) PCT No.: PCT/US14/57731

§ 371 Date: Mar. 25, 2016

Related U.S. Application Data
(60) Provisional application No. 61/882,682, filed on Sep. 26, 2013.

(51) Int. Cl. C12P 17/04; C12N 9/26

(52) U.S. Cl. CPC C12P 17/04; C12N 9/2431; C12Y 302/01026

(57) ABSTRACT

The present disclosure provides for methods of producing hydroxymethylfurfural (HMF) from fructose and other carbohydrates, novel invertase enzymes capable of catalyzing the dehydration of fructose to HMF, and methods of making the novel invertase enzymes.

### CROSS-REFERENCE TO RELATED APPLICATIONS

This application claims priority to U.S. provisional applications entitled, “Methods and Enzymes for Producing Hydroxymethylfurfural” having Ser. No. 61/882,682, filed on Sep. 26, 2013, which is entirely incorporated herein by reference.

### SEQUENCE LISTING

This application contains a sequence listing filed in electronic form as an ASCII.txt file entitled 02086586.txt, created on Sep. 26, 2014, and having a size of 7,741 bytes. The content of the sequence listing is incorporated herein in its entirety.

### BACKGROUND

Furans, such as hydroxymethylfurfural (HMF) and dimethyl furan (DMF), rank in the Department of Energy “Top 10+4” list of biobased products. (Bozell, 2010) These compounds can be produced from biomass-derived carbohydrate feedstocks by dehydration chemistry. (FIG. 1A) They provide an alternate pipeline of critical commodity materials (FIG. 1B) for use in transportation and manufacturing. For example, they have potential use as fuels, additives and polymers. Such alternates become important for consideration in cases of uncertainty in traditional supplies, prices, or in cases of extreme environments where it could be impractical to transport feed or fuelstocks.

HMF can be converted into dimethylfuran, which has a higher octane than ethanol and only slightly lower than gasoline. Further, as a platform chemical, HMF can be used to produce many other industrially important compounds and provides many potential applications, including as a precursor for a number of other materials, including, but not limited to plastics.

Conversion of fructose and other monosaccharides derived from biomass to furfurals has been known for a century and can be achieved by a variety of harsh chemical methods, typically performed in strongly acidic conditions and at high temperatures. (Lopes de Souza, 2012). However, these methods have several drawbacks, including the harsh conditions, caustic reactants, low optimization and product instability. The current methods are not environmentally friendly due to the use of extremes in pH and temperature, organic solvents, and/or potentially toxic chemical catalysts. Since current hydrocarbon resources are limited and becoming more expensive, alternative hydrocarbon sources and environmentally friendly production methods would offer advantages.

### SUMMARY

Briefly described, embodiments of the present disclosure provide methods of producing hydroxymethylfurfural (HMF) from fructose and other carbohydrates, novel enzymes capable of catalyzing the dehydration of fructose to HMF, expression vectors and transformed cells including the enzymes capable of producing HMF, and methods of making the novel enzymes.

The present disclosure provides methods of producing hydroxymethylfurfural (HMF). In embodiments the methods include providing a carbohydrate composition containing fructose, one or more carbohydrates that can be converted to fructose, or a combination thereof; exposing the carbohydrate composition to an enzyme composition including at least one isolated enzyme capable of catalyzing dehydration of fructose to HMF; and recovering the HMF produced by reaction of the fructose and the enzyme.

In embodiments, the present disclosure also provides enzymes capable of catalyzing dehydration of fructose to produce hydroxymethylfurfural (HMF). Embodiments of such enzymes include a functional variant of a native invertase protein capable of catalyzing dehydration of fructose to HMF, the variant including at least one engineered mutation relative to the native invertase. In embodiments, the variant also has a least one improvement with respect to the native invertase, such as, but not limited to, increased activity in catalyzing the dehydration of fructose to HMF, increased stability, increased rate of catalysis of fructose to HMF, and increased efficiency.

The present disclosure also provides fusion proteins capable of catalyzing production of hydroxymethylfurfural (HMF) from glucose. In embodiments, the fusion protein includes an invertase enzyme capable of catalyzing dehydration of fructose to HMF and a xylose isomerase enzyme capable of catalyzing conversion of glucose to fructose.

Embodiments of the present disclosure also include methods of making a novel invertase capable of catalyzing dehydration of fructose to hydroxymethylfurfural (HMF). In embodiments, the method includes at least the following steps: selecting a native invertase protein capable of catalyzing dehydration of fructose to HMF; creating a library of variants of the native invertase protein, each variant having at least one mutation with respect to the native protein; selecting functional variants from the library; and testing selected functional variants for improvements with respect to the native protein. In embodiments, the improvements are selected from increased activity in catalyzing the dehydration of fructose to HMF, increased stability, increased rate of catalysis of fructose to HMF, and increased efficiency.

Methods of producing hydroxymethylfurfural (HMF) according to the present disclosure, in embodiments, can include providing a cell transformed with a vector, the vector including an exogenous nucleic acid molecule encoding an invertase enzyme capable of catalyzing dehydration of fructose to HMF and a promoter operatively linked to the nucleic acid molecule such that the invertase enzyme is expressed in the cell into which it is transformed, where expression of the invertase enzyme in the cell provides the cell with the ability to produce HMF from fructose, glucose, or both, and contacting the cell with a composition including fructose, glucose, or both.

The present disclosure provides transformed cells including an expression vector including an exogenous nucleic acid molecule encoding an invertase enzyme capable of catalyzing dehydration of fructose to hydroxymethylfurfural (HMF) and a promoter operatively linked to the nucleic acid molecule encoding the invertase enzyme, such that the invertase enzyme is expressed in the cell into which it is transformed and where expression of the invertase enzyme in the cell provides the cell with the ability to produce HMF from fructose, glucose, or both.

In embodiments, the present disclosure also provides a synthesized nucleic acid molecule having the sequence of SEQ ID NO: 3. The present disclosure also provides expression vectors including a nucleic acid molecule encoding a polypeptide having the sequence of SEQ ID NO: 1 or a variant of SEQ ID NO: 1 having the mutation D138A. In embodiments, the present disclosure also provides expression vectors encoding a fusion polypeptide, where the fusion polypeptide includes an invertase enzyme capable of catalyzing dehydration of fructose to HMF operatively linked to a xylose isomerase enzyme capable of catalyzing conversion of glucose to fructose.

Other methods, compositions, plants, features, and advantages of the present disclosure will be or become apparent to one with skill in the art upon examination of the following drawings and detailed description. It is intended that all such additional compositions, methods, features, and advantages be included within this description, and be within the scope of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings. Moreover, in the drawings, like reference numerals designate corresponding parts throughout the several views.

FIG. 1A illustrates the dehydration of a carbohydrate to produce hydroxymethylfurfural (HMF), and FIG. 1B illustrates HMF as a platform compound for the production of (as described in Rosatella, et al., incorporated herein by reference).

FIG. 2 illustrates a series of reaction schemes showing possible pathways to HMF and decomposition products from fructose under strongly acidic catalysis. The top half shows the cyclic pathway, the bottom panel presents the acyclic pathway. Either pathway may produce levulinic acid from HMF, with the acyclic path producing unique side products. (Roman-Leshkov et al., 2006, incorporated herein by reference).

FIG. 3 illustrates a series of reaction schemes showing acid catalyzed cyclic pathways for generation of HMF and side products from fructose, with the boxed region as the productive route to HMF (Akien, et al. 2012, incorporated herein by reference)

FIG. 4 illustrates a reaction scheme illustrating invertase-catalyzed dehydration of fructose en route to HMF, showing a stepwise generation of an oxocarbenium ion intermediate.

FIG. 5 is a graph illustrating the kinetic characterization of native invertase and D138A mutant invertase. Each time point represents a triplicate determination of the amount of HMF formed using the HPLC assay. Triangles (bottom) represent the background reaction without any invertase; diamonds (middle) represent data obtained with native (wt) invertase; and squares (top) represent data obtained for rate of reaction for the D138A mutant invertase.

FIG. 6 illustrates the structure of the active site of invertase with bound fructose.

FIG. 7 illustrates the relative position of active site residues making contact with bound substrate (fructose), with the residues representing initial targets for saturation mutagenesis.

FIGS. 8A and 8B illustrate the crystal structure of invertase from T. maritime with bound D-fructose. FIG. 8A shows the overall structure of one of six invertase monomers, with one molecule of D-fructose in licorice format bound in the active site beta-propeller domain. FIG. 8B is a close up view of the active site with bound D-fructose. Relevant catalytic residues are labeled, with the electron density map shown in grey.

FIG. 9 is a schematic diagram illustrating library design, screening, and analysis for invertase mutant libraries.

FIG. 10 is an electropherogram illustrating the sequence analysis of the D138X mutagenesis library a demonstrating good coverage of NNK codon space.

FIG. 11 illustrates the two step conversion of glucose to HMF, or a mixture of glucose and xylose to HMF and furfural.

FIG. 12 illustrates HPLC analysis of isotope effects for conversion of fructose to HMF.

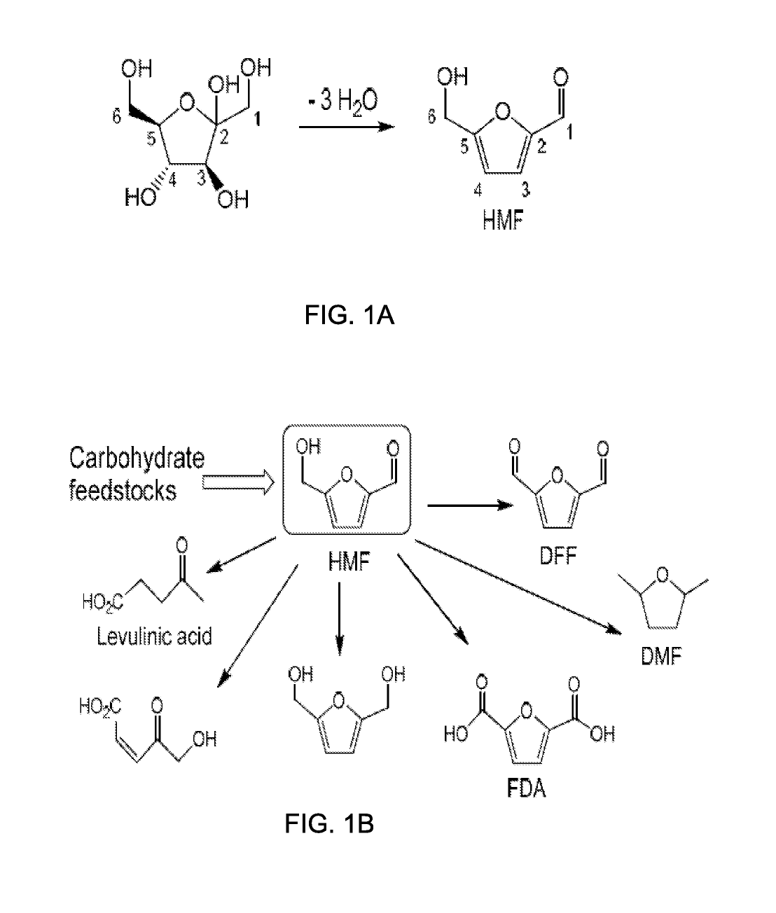

### DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

Any publications and patents cited in this specification that are incorporated by reference are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biology, biochemistry, molecular biology, proteomics, and the like, which are within the skill of the art. Such techniques are explained fully in the literature.

It must be noted that, as used in the specification and the appended embodiments, the singular forms “a,” “an,” and “the” include plural referents unless the context clearly dictates otherwise. Thus, for example, reference to “a cell” includes a plurality of cells. In this specification and in the embodiments that follow, reference will be made to a number of terms that shall be defined to have the following meanings unless a contrary intention is apparent. Throughout this application, the term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and “and/or.”

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps. “Consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes any prior art embodiments.

Prior to describing the various embodiments, the following definitions are provided and should be used unless otherwise indicated.

### DEFINITIONS

In describing the disclosed subject matter, the following terminology will be used in accordance with the definitions set forth below.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” Representative examples of the nucleic acids of the present disclosure include bacterial plasmid vectors including expression, cloning, cosmid and transformation vectors such as, but not limited to, pBR322, animal viral vectors such as, but not limited to, modified adenovirus, influenza virus, polio virus, pox virus, retrovirus, insect viruses (baculovirus), and the like, vectors derived from bacteriophage nucleic acid, and synthetic oligonucleotides like chemically synthesized DNA or RNA. The term “nucleic acid” further includes modified or derivatized nucleotides and nucleosides such as, but not limited to, halogenated nucleotides such as, but not only, 5-bromouracil, and derivatized nucleotides such as biotin-labeled nucleotides.

As used herein, “isolated” means removed or separated from the native environment. Therefore, isolated DNA can contain both coding (exon) and noncoding regions (introns) of a nucleotide sequence corresponding to a particular gene. An isolated peptide or protein indicates the protein is separated from its natural environment. Isolated nucleotide sequences and/or proteins are not necessarily purified. For instance, an isolated nucleotide or peptide may be included in a crude cellular extract or they may be subjected to additional purification and separation steps.

With respect to nucleotides, “isolated nucleic acid” refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example but not limited to, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, e.g., a gene encoding a fusion protein, and (e) a recombinant nucleotide sequence that is part of a hybrid sequence that is not naturally occurring. Isolated nucleic acid molecules of the present disclosure can include, for example, natural allelic variants as well as nucleic acid molecules modified by nucleotide deletions, insertions, inversions, or substitutions.

It is advantageous for some purposes that a nucleotide sequence is in purified form. The term “purified” in reference to nucleic acid represents that the sequence has increased purity relative to the natural environment.

The terms “polynucleotide,” “oligonucleotide,” and “nucleic acid sequence” are used interchangeably herein and include, but are not limited to, coding sequences (polynucleotide(s) or nucleic acid sequence(s) which are transcribed and translated into polypeptide in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)). No limitation as to length or to synthetic origin is suggested by the terms described herein.

The term “fragment” as used herein to refer to a nucleic acid (e.g., cDNA) refers to an isolated portion of the subject nucleic acid constructed artificially (e.g., by chemical synthesis) or by cleaving a natural product into multiple pieces, using restriction endonucleases or mechanical shearing, or a portion of a nucleic acid synthesized by PCR, DNA polymerase or any other polymerizing technique well known in the art, or expressed in a host cell by recombinant nucleic acid technology well known to one of skill in the art. The term “fragment” as used herein may also refer to an isolated portion of a polypeptide, wherein the portion of the polypeptide is cleaved from a naturally occurring polypeptide by proteolytic cleavage by at least one protease, or is a portion of the naturally occurring polypeptide synthesized by chemical methods well known to one of skill in the art.

The term “gene” or “genes” as used herein refers to nucleic acid sequences (including both RNA and DNA) that encode genetic information for the synthesis of a whole RNA, a whole protein, or any portion of such whole RNA or whole protein. Genes that are not naturally part of a particular organism's genome are referred to as “foreign genes,” “heterologous genes” or “exogenous genes” and genes that are naturally a part of a particular organism's genome are referred to as “endogenous genes”. The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by “foreign, heterologous or exogenous genes” and are, therefore, not naturally expressed in the cell.

The term “polypeptides” and “protein” include proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. In addition, the term “variant” as used herein includes mutations of proteins and peptides, whether such mutations occur naturally or by human design.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein “functional variant” refers to a variant of a protein or polypeptide (e.g., a variant of a isomerase protein) that can perform the same functions or activities as the original protein or polypeptide, although not necessarily at the same level (e.g., the variant may have enhanced, reduced or changed functionality, so long as it retains the basic function).

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

The term “expression” as used herein describes the process undergone by a structural gene to produce a polypeptide. It is a combination of transcription and translation. Expression generally refers to the “expression” of a nucleic acid to produce a polypeptide, but it is also generally acceptable to refer to “expression” of a polypeptide, indicating that the polypeptide is being produced via expression of the corresponding nucleic acid.

The term “plasmid” as used herein refers to a non-chromosomal double-stranded DNA sequence including an intact “replicon” such that the plasmid is replicated in a host cell.

As used herein, the term “vector” or “expression vector” is used in reference to a vehicle used to introduce an exogenous nucleic acid sequence into a cell. A vector may include a DNA molecule, linear or circular, which includes a segment encoding a polypeptide of interest operably linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, the term “promoter” or “promoter region” includes all sequences capable of driving transcription of a coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

The term “operably linked” indicates that the regulatory sequences for expression of the coding sequences of a nucleic acid are placed in the nucleic acid molecule in the appropriate positions relative to the coding sequence so as to effect expression of the coding sequence. This same definition is sometimes applied to the arrangement of coding sequences and transcription control elements (e.g. promoters, enhancers, and termination elements), and/or selectable markers in an expression vector.

As used herein, the term “selectable marker” refers to a gene whose expression allows one to identify cells that have been transformed or transfected with a vector containing the marker gene. For instance, a recombinant nucleic acid may include a selectable marker operably linked to a gene of interest and a promoter, such that expression of the selectable marker indicates the successful transformation of the cell with the gene of interest.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the term “transfection” refers to the introduction of an exogenous nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome. A “transformed” cell is thus a cell transfected with a nucleic acid sequence. The term “transformation” refers to the introduction of a nucleic acid (e.g., DNA or RNA) into cells in such a way as to allow expression of the coding portions of the introduced nucleic acid. The term “transgene” refers to an artificial gene which is used to transform a cell of an organism, such as a bacterium or a plant.

The term “recombinant” generally refers to a non-naturally occurring nucleic acid, nucleic acid construct, or polypeptide. Such non-naturally occurring nucleic acids include natural nucleic acids that have been modified, for example that have deletions, substitutions, inversions, insertions, etc., and combinations of nucleic acid sequences of different origin that are joined using molecular biology technologies (e.g., a nucleic acid sequences encoding a “fusion protein” (e.g., a protein or polypeptide formed from the combination of two different proteins or protein fragments)), the combination of a nucleic acid encoding a polypeptide to a promoter sequence, where the coding sequence and promoter sequence are from different sources or otherwise do not typically occur together naturally). Recombinant also refers to the polypeptide encoded by the recombinant nucleic acid. Non-naturally occurring nucleic acids or polypeptides include nucleic acids and polypeptides modified by man.

The terms “native,” “wild type”, or “unmodified” polypeptide, protein or enzyme, are used herein to provide a reference point for a variant/mutant of a polypeptide, protein, or enzyme prior to its mutation and/or modification (whether the mutation and/or modification occurred naturally or by human design). Typically, the unmodified, native, or wild type polypeptide, protein, or enzyme has an amino acid sequence that corresponds substantially or completely to the amino acid sequence of the polypeptide, protein, or enzyme as it generally occurs naturally or in vivo.

An “enzyme,” as used herein, is a polypeptide that acts as a catalyst, which facilitates and generally speeds the rate at which chemical reactions proceed but does not alter the direction or nature of the reaction.

As used herein, the term “improvement” or “enhancement” generally refers to a change or alteration in a function or behavior of a protein, such as an enzyme, that in the applicable circumstances is considered to be desirable.

As used herein, the term “enhance,” “increase,” and/or “augment” generally refers to the act of improving a function or behavior relative to the natural, expected or average. For example, a mutated (e.g., variant) protein that has increased activity over that of the corresponding native/wild type protein, can have improved activity (e.g. a faster rate of reaction, or binding/reacting with a greater number of substrates in the same amount of time) as compared to the activity of the corresponding wild type protein. Similarly, a protein with increased stability has a longer half life, is more resistant to degradation, and/or is less sensitive to changes in various environmental conditions, etc. Increased rate of reaction for an enzyme of the present disclosure indicates, e.g., that the enzyme catalyzes the reaction faster than a corresponding wild type protein, and increased efficiency indicates, e.g., that an enzyme has a more efficient reaction (in terms of time, energy, waste, etc.) than a corresponding wild type enzyme, which can be determined by tests known in the art.

The term “conformation” in reference to a protein or peptide (e.g. “folded conformation”) generally refers to the higher folded states of the peptide beyond the primary structure (peptide sequence), particularly to the tertiary structure of the protein or peptide.

An “insertion” or “addition”, as used herein, refers to a change in an amino acid or nucleotide sequence resulting in the addition or insertion of one or more amino acid or nucleotide residues, respectively, as compared to the corresponding naturally occurring molecule.

A “deletion” or “subtraction”, as used herein, refers to a change in an amino acid or nucleotide sequence resulting in the deletion or subtraction of one or more amino acid or nucleotide residues, respectively, as compared to the corresponding naturally occurring molecule.

A “substitution”, as used herein, refers to the replacement of one or more amino acids or nucleotides by different amino acids or nucleotides, respectively.

A “mutation” is a heritable change in genetic material, usually relative to a reference “wild-type” DNA sequence. Mutations can occur as a result of a single base change, multiple base changes, or the addition or deletion of more than one nucleotide to a DNA sequence, or rearrangement of larger sections of genes or chromosomes. Mutations can occur naturally or by human intervention and/or design. An “engineered mutation” refers to a mutation created by human design (e.g., the mutation did not spontaneously occur by natural causes and/or was the result of intentional human manipulation). A “genetically modified” organism is an organism whose genetic material has been altered by one or more engineered mutations (e.g., human induced mutations).

The term “mutant” is employed broadly to refer to a protein that differs in some way from a reference wild-type protein, where the protein may retain biological properties of the reference wild-type (e.g., naturally occurring) protein, or may have biological properties that differ from the reference wild-type protein. A mutant protein is a variant (likewise, a functional mutant is a functional variant if native function is retained, although its function may be reduced or enhanced). The term “biological property” of the subject proteins includes, but is not limited to, function/activity, rate of reaction, structural conformation, and the like; in vivo and/or in vitro stability (e.g., half-life); and the like. Mutants can include single amino acid changes (point mutations), deletions of one or more amino acids (point-deletions), N-terminal truncations, C-terminal truncations, insertions, and the like. Mutants can occur in nature and/or be generated using standard techniques of molecular biology.

The term “invertase” as used in the present disclosure indicates a protein from the class of invertase enzymes capable of catalysis of carbohydrates to HMF. As used herein, “β-fructofuranosidase” refers to a class of invertase enzymes, such as, but not limited to, β-fructofuranosidase from Thermatoga maritime having the peptide sequence of SEQ ID NO: 1 and the naturally occurring functional variants/homologs of SEQ ID NO: 1. In the present disclosure, “β-fructofuranosidase” includes the protein of SEQ ID NO: 1 as well as functional variants and/or homologs (e.g., orthologs and paralogs) thereof retaining the function catalysis of fructose to HMF. Invertases in the present disclosure can include invertases from the glycohydrolase family 32 (GH32) capable of catalysis of carbohydrates to HMF. Although it is known that even low homology sequences can have the same or similar functions due to folding and location of the active site of the catalyst (e.g., similar tertiary structure), in embodiments “β-fructofuranosidase” includes proteins having a sequence of SEQ ID NO: 1 as well as variants of SEQ ID NO: 1 having about 50% or more, 60% or more, about 70% or more, about 80% or more, about 90% or more, about 95% or more, or about 99% or more (including any intervening ranges) sequence identity with SEQ ID NO: 1. For instance, in embodiments a β-fructofuranosidase in the present disclosure includes variants of SEQ ID NO: 1 having a mutation (e.g., a substitution) of one or more of the following amino acids: N16, D17, Q33, W41, F74, S75, R137, D138 (e.g., D138A), E190, Y240, A241, and W260.

### DISCUSSION

The embodiments of the present disclosure encompass methods and compositions for producing hydroxymethylfurfural (HMF) from carbohydrates. In specific embodiments, the disclosure includes methods and compositions for converting sugars to HMF by enzyme catalysis. Embodiments include methods and compositions for producing HMF from fructose and/or from other sugars that can be converted to fructose. The present disclosure also provides novel enzymes for catalyzing the chemical reaction of fructose to HMF and biological systems for producing HMF using enzyme catalysis.

The present disclosure provides new methods for production of HMF from sugars or other carbohydrates by enzyme catalysis as well as new enzymes for use in this process. The methods and compositions of the present disclosure provide ways to produce important platform chemicals from sugars in an environmentally friendly manner, as opposed to methods currently in use that employ acids, metal catalysis, organic solvents, and harsh process conditions.

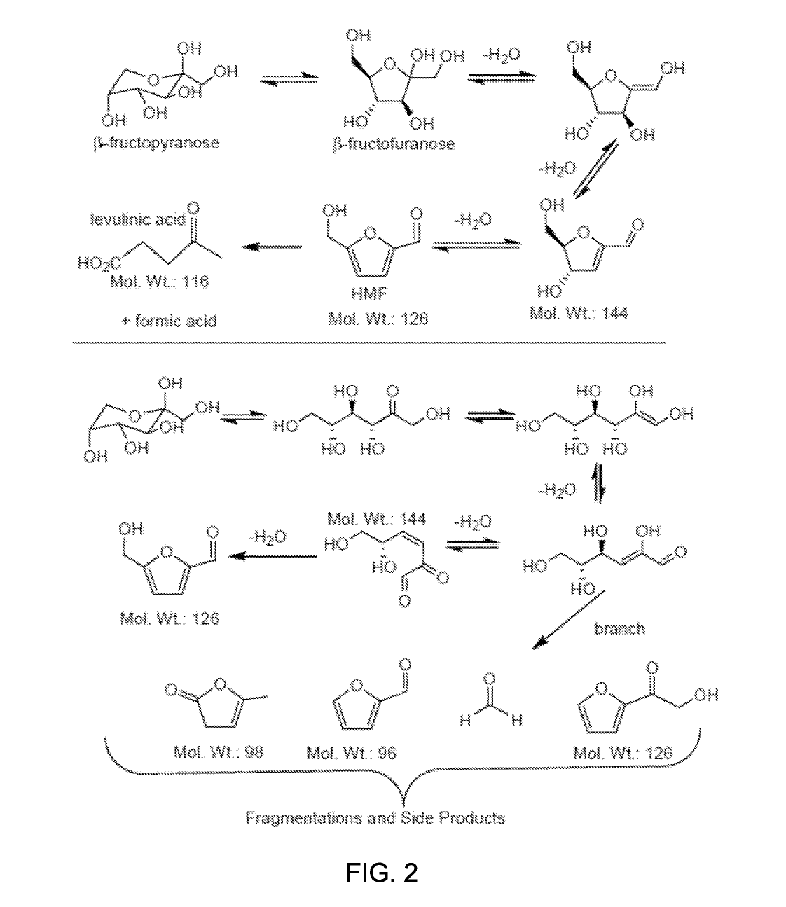

The conventional processes (illustrated in FIG. 2) can suffer from side reactions, and HMF can decompose to levulinic acid (Rosatella, 2001; van Putten, 2013). FIG. 2 presents two commonly proposed mechanism for HMF production: one being the “cyclic” route, proceeding via a furanosyl ring, and the other an acyclic mechanism involving a series of acyclic intermediates that cyclize and finally dehydrate to form HMF. Progress is being made in process optimization to minimize side reactions and product instability (Binder, 2009); however, these chemical processes require metal-based catalysts and/or process conditions that are not environmentally “green”. These HMF production systems are not biocompatible, and do not avoid the need for extremes of pH and/or exotic catalysts and organic solvents.

The present disclosure provides enzymatic systems that produce HMF, with the attendant benefits of an inexpensive, renewable catalyst that works in water and is biocompatible and environmentally friendly. Further, such a catalyst can be incorporated into a metabolic pathway in a simple microorganism such that a fermentative route from biomass to HMF or derivatives could be developed. It is believed that enzymatic production of HMF has not been previously reported.

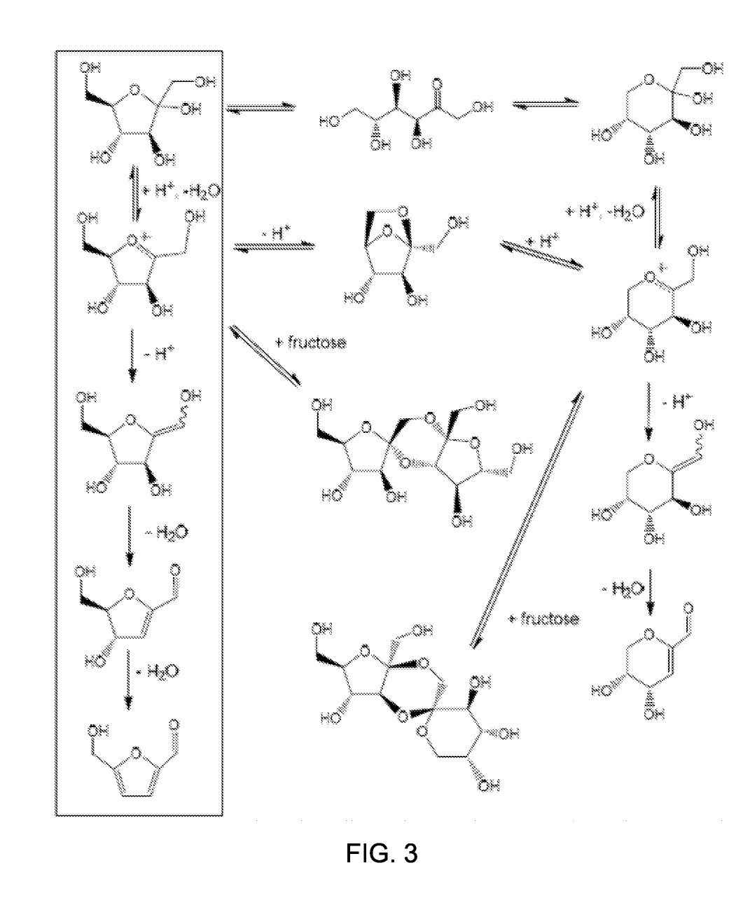

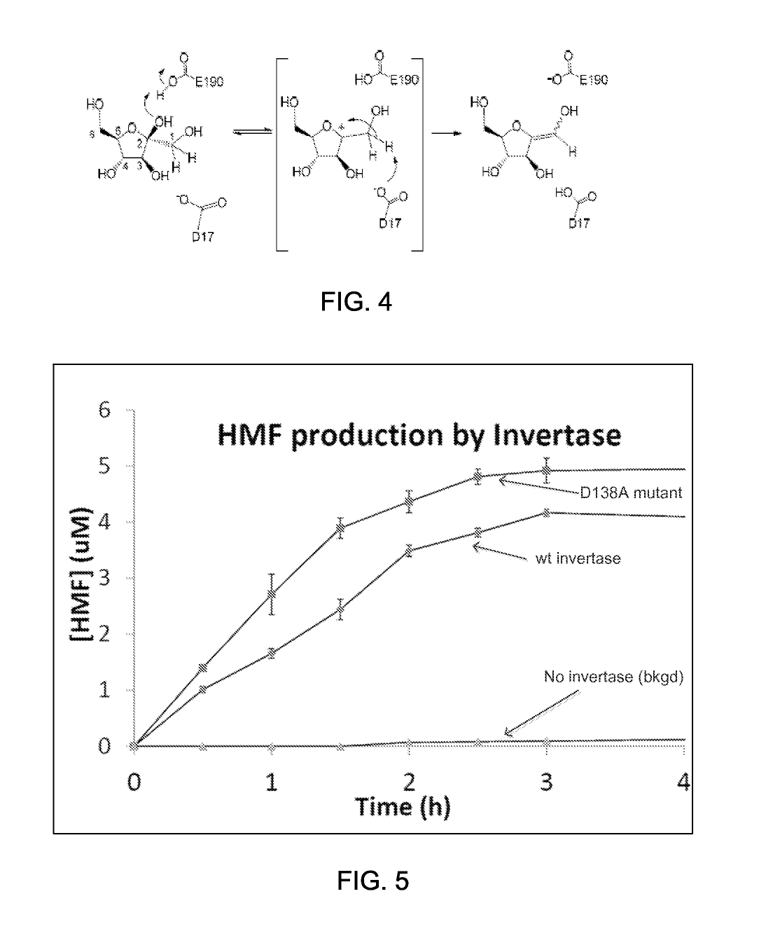

Although dehydration of fructose to HMF is not a transformation developed by nature, acid and/or base catalysis reactions in nature represent a chemical imperative of catalyzing a dehydration reaction. The mechanism for formation of HMF from fructose as discussed by Akien (Akien, et al. 2012) involves generation of a fructosyl oxocarbenium ion. (FIG. 3). As such, an enzyme capable of generating a fructosyl oxocarbenium ion might be capable of catalyzing HMF from fructose. An example of enzymatic machinery for performing this task is a glycosidase, (Voclado, 2008), such as, but not limited to, fructose-recognizing glycosidases. One such glycosidase includes glycosidase E.C.#3.2.1.26, otherwise known as invertase or β-fructofuranosidase. The invertase from the hyper-thermophile Thermatoga maritima is a highly thermostable catalyst and was identified as a starting point for engineering a new activity while maintaining thermostability. β-fructofuranosidase from T. maritima has a protein sequence of SEQ ID NO: 1, (and including any known variants which includes organism-specific codon-optimized variant). β-fructofuranosidase from T. maritima is encoded by the nucleic acid sequence having SEQ ID NO: 2 as well as the synthetic, codon-optomized nucleic acid sequence having SEQ ID NP: 3. The enzyme has a broad pH profile accompanied by an excellent thermostability, with 41% activity remaining after incubation at 60° C. for 14 days. (Liebl, et al. 1998) This invertase has a high resolution crystal structure (PDB 1W2T) available (Alberto, et al. 2006) and it uses the retaining mechanism for catalysis (Vocadlo, 2008), involving two active site residues, D17 and E190. The mechanism (FIG. 4) involves E190 protonating the glycosidic oxygen to generate an oxocarbenium ion-like transition state that is transiently captured by the nucleophile D17.

The present disclosure, as described in the examples below, demonstrates that this same catalytic machinery can facilitate HMF formation by using acid/base catalysis to protonate the O₂ hydroxyl of bound β-fructofuranose, generating an oxocarbenium ion transition state that could then partition into the enolic elimination product as shown in FIG. 4. As shown in FIG. 3, the initial dehydration of fructose and deprotonation represent essential steps for conversion to HMF. Branching in the mechanism, prior to these steps leads to unwanted non-HMF degradation products. By catalyzing the HMF forming sequence in the active site of the enzyme, at pH values closer to neutrality, it is believed that the issues of pathway branching and product degradation can be controlled by accelerating the first irreversible step (Akien, 2012) while at the same time employing mild conditions in which the product is stable.

As described in greater detail in the Examples below, the present disclosure demonstrates that some enzymes are capable of catalyzing production of HMF from fructose. Thus, methods of the present disclosure include methods of producing HMF by exposing a carbohydrate composition that includes fructose to an enzyme capable of catalyzing dehydration of fructose to produce HMF. In embodiments, the enzyme, or enzyme composition, includes a glycosidase that is capable of catalyzing the dehydration reaction of fructose or other carbohydrates to HMF. In embodiments the enzyme is a member of the glycohydrolase family 32 (GH32) capable of catalyzing the dehydration reaction of fructose or other carbohydrates to HMF. Enzymes capable of catalyzing the dehydration of fructose to HMF include, but are not limited to, glycosides from the glycohydrolase family 32. In embodiments, the enzyme is an invertase, such as, but not limited to, β-fructofuranosidase from T. maritima.

In embodiments the enzyme has the protein sequence of SEQ ID NO: 1 and functional variants of SEQ ID NO: 1. While it is scientifically understood that protein sequences with low sequence similarity (e.g., 25%) can still have similar functions due to possible similarities in tertiary structure (in spite of sequence differences). Although some members of the glycohydrolase family 32 may have lower sequence similarity to SEQ ID NO: 1 while still possessing the capability of catalysis of carbohydrates to produce HMF, in some embodiments of the present disclosure the enzyme is a functional variant of SEQ ID NO: 1 having 50% or more sequence identity to SEQ ID NO: 1.

In some embodiments, the enzyme of the present disclosure is a β-fructofuranosidase enzyme encoded by the nucleic acid sequence of SEQ ID NO: 2, and homologs thereof. In embodiments, the enzyme is a β-fructofuranosidase enzyme encoded by the non-naturally occurring (e.g., engineered/synthesized) nucleic acid sequence of SEQ ID NO: 3, which is a codon optimized sequence encoding a β-fructofuranosidase enzyme of the present disclosure. The present disclosure also includes an isolated nucleic acid molecule having SEQ ID NO: 3.

Functional variants of native enzymes, such as β-fructofuranosidase from T. maritima, can also be used in the methods of the present disclosure. Functional variants can be identified by generating mutants of native enzymes (e.g., invertases) and screening for retained and/or improved activities or features. For instance, in embodiments of the methods of producing HMF of the present disclosure, the enzyme is a functional variant of an invertase, such as, but not limited to, a variant of β-fructofuranosidase from T. maritima, where the variant has at least one mutation from the native enzyme, at least one improvement over the native β-fructofuranosidase. In embodiments the improvement can be selected from improvements such as, but not limited to, increased activity, increased stability, increased rate of catalysis, and increased efficiency. In embodiments, the functional variant has an improvement in the level, rate, or efficiency of the reaction, while maintaining stability at substantially the same level as the native protein.

In embodiments, the carbohydrate is fructose, but it can include other sugars, such as glucose. In embodiments, glucose can be converted to fructose by an enzyme, such as but not limited to, an isomerase. Thus, in embodiments, an enzyme composition may include not only the enzyme capable of catalyzing dehydration of fructose to HMF but also include a second enzyme capable of converting glucose, or other monosaccharide, to fructose. In embodiments, the enzyme for converting glucose to fructose is xylose isomerase. In embodiments a carbohydrate composition is first contacted with an isomerase to convert other carbohydrates to fructose and then the fructose is exposed to an enzyme to convert the fructose to HMF, and in other embodiments the carbohydrate composition including glucose, fructose, or a combination is contacted with both enzymes in the same mixture. In other embodiments, the two enzymes may be coupled together to provide a fusion enzyme that can catalyze both reactions simultaneously. In an embodiment, xylose isomerase is coupled to an invertase, such that the coupled enzyme can produce fructose from glucose as well as producing HMF from fructose, thus allowing in situ conversion of glucose to HMF. In embodiments, the xylose isomerase and/or the fusion enzyme described above can also produce furfural from xylose.

In embodiments the method of the present disclosure of producing HMF is performed at a pH range of about 4.0 to about 8.0. In embodiments it is at a pH of about 4.5 to about 6.0. In embodiments the method is performed at a temperature of about 40° C. to about 75° C. In embodiments, the temperature is about 50° C. to about 65° C. Additional descriptions of the methods of producing HMF of the present disclosure are provided in the detailed examples below.

The present disclosure also provides novel enzymes capable of catalyzing conversion of carbohydrates to HMF and methods of designing and identifying novel enzymes capable of catalyzing the production of HMF from fructose or other sugars. In an embodiment, one or more native enzymes capable of catalyzing dehydration of fructose to HMF are identified. Then mutagenesis is conducted on the native enzyme to produce a library of variants. Each variant in the library includes at least one mutation with respect to the native enzyme. The mutagenesis can be random or directed mutagenesis, or a combination. The variant library is then screened for functional variants. Then the functional variants are further screened for improvements relative to the native protein. Improvements include, but are not limited to: increased activity, increased stability, increased rate of catalysis, and increased efficiency. Novel invertase variants with desired improvements can be selected for use in production of HMF and/or for further characterization and/or optimization, such as described in the examples below. In embodiments, crystal structure of a subset of selected variants is obtained, and additional characterization of physical conformation and activity is conducted. After further characterization, additional mutagenesis may be implemented to obtain further enhanced variants. Embodiments of such methods are described in greater detail in the examples below.

The present disclosure further provides novel enzymes capable of catalyzing production of HMF from carbohydrates, including sugars, such as but not limited to, fructose and sucrose. The present disclosure also includes isolated and/or synthesize/engineered nucleic acids encoding the enzymes of the present disclosure. In embodiments, such novel enzymes are mutants of native enzymes, such as invertases, where the mutants retain the native activity (e.g., functional variants) and/or include improvements over the native enzyme. Such improvements can include, but are not limited to: increased activity in converting fructose, etc., to HMF, increased stability, increased rate of catalysis of fructose, etc., to HMF, and increased efficiency. In addition, some mutants may include the ability to catalyze formation of HMF from different substrates than the native enzyme. For instance, if the native enzyme catalyzes production of HMF from fructose, the improved enzyme may produce HMF from glucose, or both fructose and glucose. In embodiments, the functional variant is capable of catalyzing the dehydration of fructose to HMF, performs this reaction catalysis at a greater rate, yield, or efficiency than the native protein and retains the same stability as the native protein.

In embodiments of the present disclosure, the enzyme capable of producing HMF from fructose comprises a functional variant of β-fructofuranosidase from T. maritima. Due to determination of the crystal structure of the native β-fructofuranosidase while bound to substrate, certain active site residues have been identified as candidates for directed mutagenesis. Thus, in embodiments, the native invertase is T. maritima 6-fructofuranosidase (e.g., SEQ ID NO: 1 and natural variants thereof), and the variant has been engineered to have a mutation (e.g., point mutation, substitution, deletion, etc.) involving one or more of the following amino acid residues of the native enzyme: N16, D17, Q33, W41, F74, S75, R137, D138, E190, Y240, A241, and W260. As described in greater detail in the examples below, saturation mutagenesis of D138 resulted in a library of mutants D138X (where X indicates the aspartic acid at position 138 of the protein sequence (SEQ ID NO: 1) is replaced with “X”, representing any other amino acid). Some of the D138X mutants had improved activity and/or stability over wild type β-fructofuranosidase, including D138A. In embodiments the mutant β-fructofuranosidase has the mutation D138A. Embodiments of the present disclosure also include a nucleic acid sequence encoding the mutant D138A invertase.

In further embodiments, the present disclosure includes a fusion protein including an enzyme, such as T. maritima β-fructofuranosidase or functional variants thereof, as described above, coupled to a xylose isomerase, such that the fusion protein is capable of catalyzing the conversion of glucose to fructose and then producing HMF from fructose, thereby enabling production of HMF from glucose. In embodiments, the fusion protein is also capable of catalyzing the conversion of xylose into furfural. The present disclosure also includes nucleic acids encoding fusion proteins of the present disclosure as well as vectors including the nucleic acids encoding a fusion protein of the present disclosure.

The present disclosure also includes methods of producing HMF by transforming a cell with an expression vector including an exogenous nucleic acid molecule encoding an invertase enzyme of the present disclosure that is capable of catalyzing dehydration of fructose to HMF. The vector also includes a promoter operatively linked to the nucleic acid molecule such that the invertase enzyme is expressed in the cell into which it is transformed. Expression of the invertase enzyme in the transformed cell provides the cell with the ability to produce HMF from fructose, glucose, or both, depending on the nature of the enzyme. In embodiments of the methods of producing HMF, the transformed cell is contacted with a composition including fructose, glucose, or both (e.g., grown in a medium containing such sugars). In embodiments, the invertase enzyme is β-fructofuranosidase from T. maritima or a functional variant thereof, such as described above. In embodiments of the present disclosure, the cell is a single celled microorganism, such as, but not limited to, a bacterium. In embodiments, methods of the present disclosure include recovering the HMF and/or furfural produced by the cell.

In embodiments, the expression vector can also include a nucleic acid molecule encoding a xylose isomerase enzyme to facilitate conversion of glucose to fructose and/or xylose to furfural. In embodiments, the present disclosure also provides an expression vector encoding a fusion polypeptide, where the fusion polypeptide includes an invertase enzyme of the present disclosure capable of catalyzing dehydration of fructose to HMF operatively linked to a xylose isomerase enzyme capable of catalyzing conversion of glucose to fructose.

The present disclosure also includes transformed cells including the vector described above having an exogenous nucleic acid molecule encoding an invertase enzyme capable of catalyzing dehydration of fructose to HMF, and a promoter operatively linked to the nucleic acid molecule such that the invertase enzyme is expressed in the cell into which it is transformed.

Additional details regarding the methods and compositions of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and protected by the following embodiments.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Establishing Catalysis of Fructose to HMF by Invertase

The present example demonstrates that the same catalytic machinery of T. maritima invertase facilitates HMF formation from fructose. These results demonstrate that the enzyme catalyzes HMF formation by using acid/base catalysis to protonate the 02 hydroxyl of bound β-fructofuranose, generating an oxocarbenium ion transition state that could then partition into the enolic elimination product as shown in FIG. 4. As shown in FIG. 3 (adapted from Akien, et al. 2012, incorporated herein by reference), the initial dehydration of fructose and deprotonation represent steps for conversion to HMF. Branching in the mechanism, prior to these steps, leads to unwanted non-HMF degradation products. By catalyzing the HMF forming sequence in the active site of the invertase enzyme, at pH values closer to neutrality, it is believed that the issues of pathway branching and product degradation can be controlled by accelerating the first irreversible step while at the same time employing mild conditions in which the product is stable.

The catalytic potential of the T. maritima invertase was tested in the following way. The gene was synthesized with codon optimization (using Genewiz) and subcloned into pET28 with an N-terminal His6 tag. The enzyme was purified to homogeneity and surveyed for its ability to produce HMF with fructose at different temperatures and pH. Initial experiments showed the enzyme is able to enhance HMF production from fructose in 50 mM acetate buffer at pH 4.5 and 65° C., relative to the same reaction in the absence of enzyme. Analysis by initial velocity kinetics, 1H-NMR, and LC-ESI-MS product analysis reveals the wild-type invertase doubles the rate of HMF production.

However, a combination of high background rate and operating near the isolelectric point (pI) of the enzyme made spectrophotometric analyses troublesome due to precipitation of the invertase. At pH 6.0, sodium phosphate buffer, 50° C. the enzyme had improved solubility, and the relative rate acceleration over background was notable at approximately 60 fold (FIG. 5, middle plot). It is reasonable to consider that the enzyme has a different active site ionization profile at pH 6.0 vs. pH 4.5, which may account for the observed rate acceleration. In addition it was observed that the invertase catalyzed synthesis of HMF was accelerated by increasing amounts of invertase (data not shown).

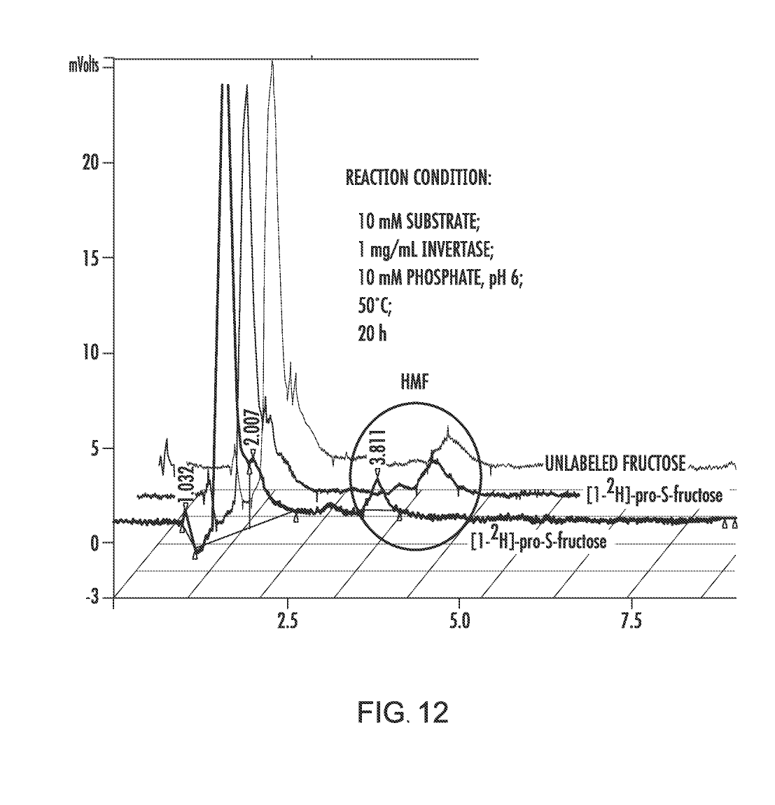

Any possible isotope effects were determined by comparison of initial rates for reactions of fructose, [1-²H]-proR fructose and [1-²H]-proS fructose (Omicron Biochemical). 10 mM concentrations of fructose, 1-²H-proR or 1-²H-proS fructose were allowed to react with 1 mg/mL invertase in pH 6.0 phosphate buffer, and the resulting HMF product was analyzed by HPLC. Evidence for a normal kinetic isotope effect was not found; integrated areas for the HMF peak were not substantially different (FIG. 12). This result indicates that the first step in the putative mechanism is not rate determining and, therefore, is found in subsequent steps.

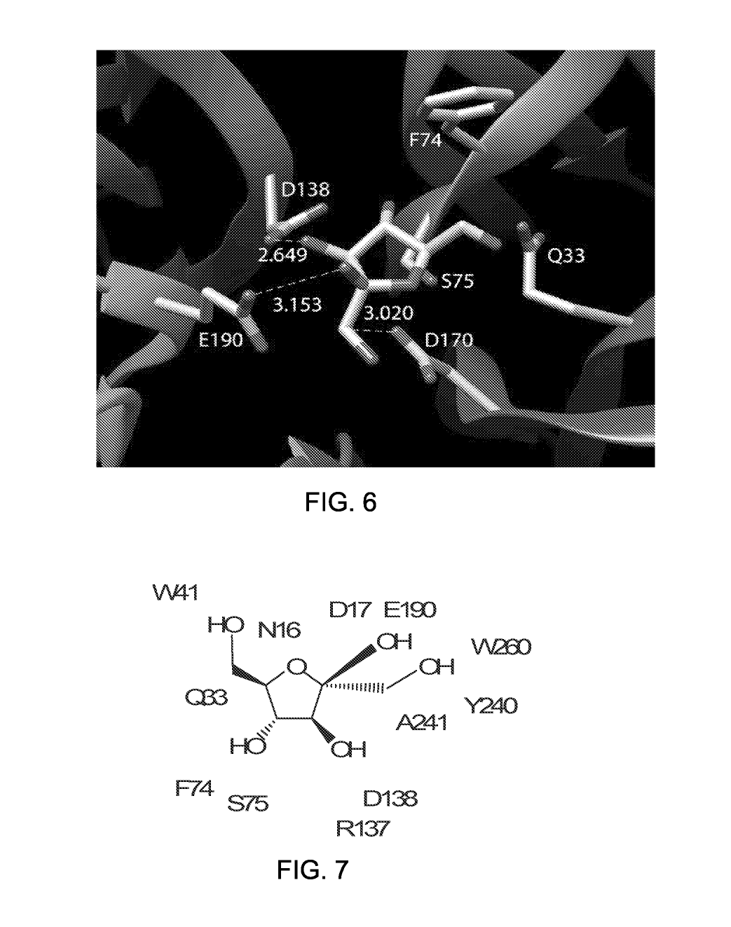

The structural biology of invertase was studied to provide additional insights into possible reaction mechanisms. Examination of the structure for invertase (FIGS. 6, 7, and 8) revealed that the active site may already position catalytic residues in appropriate places for the first two dehydrations. (Alberto et al. 2004, Alberto et al. 2006) It is possible (see FIGS. 3 and 4 for mechanism) that E190 protonates 02, then D17 deprotonates at C1 to produce the first dehydration product to the enolic intermediate. This then tautomerizes with beta-elimination of 03, catalyzed by the well placed D138. These two dehydrations would then yield the penultimate cyclopentenal (FIG. 3, left). The final dehydration step, involving loss of O4 and H5, involves aromatization and is likely to have a favorable free energy. Near the C4-C5 carbons, Q33, F74 and S75 appear well-suited targets for introduction of catalytic functionality. The final dehydration could involve a stepwise protonation of 04, loss of water, with a subsequent facile loss of a proton from C5.

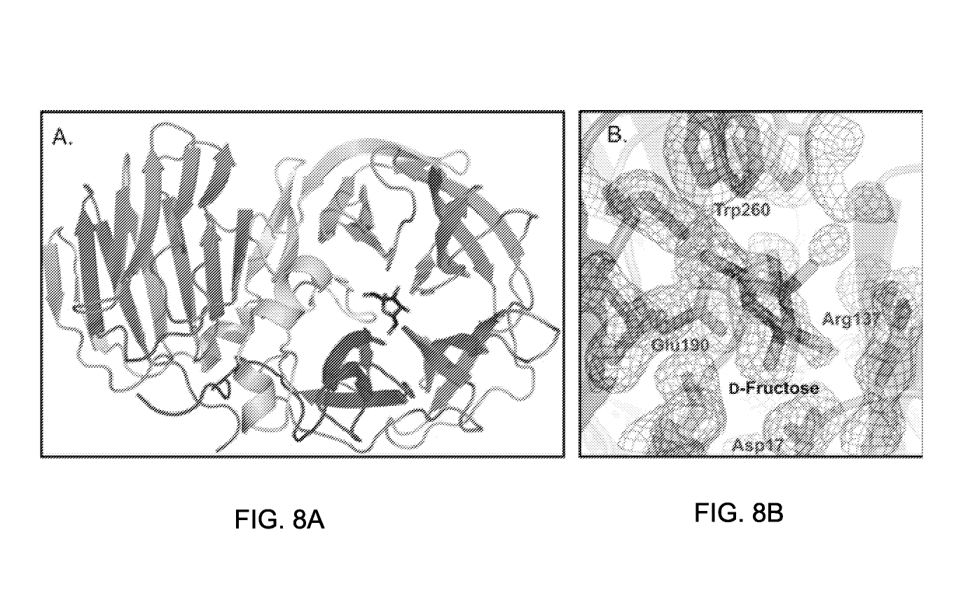

Invertase structure was also studied with crystallography. The gene for T. maritima invertase (SEQ ID NO: 2) was sequenced and a codon-optimized nucleotide sequence (SEQ ID NO: 3) was synthesized and cloned into the pET28 vector with an N-terminal hexa-histidine tag as described. The T. maritima invertase protein (SEQ ID NO: 1) was overexpressed in E. coli and purified to high homogeneity with nickel-NTA affinity, anion-exchange and size exclusion chromatography. The pure enzyme was concentrated to 20 mg/mL and dialyzed into a low salt buffer. Crystals were formed using the hanging-drop diffusion method by mixing 1 μL invertase, 1 μL 20 μM D-fructose and 2 μL of well solution containing: 11% PEG 1000, 150 mM lithium sulfate and 100 mM sodium citrate pH=4.6. Plate-like crystals formed within 7 days with dimensions approximately: 100 μm×100 μm×20 μm. Crystals were briefly soaked in well solution with 20 mM D-fructose and 10% glycerol before flash frozen in liquid nitrogen. Diffraction data was collected on a Rigaku RU-300 generator with R-AXIS IV++ imaging plate system to a resolution of 1.65 Å and a Rmerge of 4.3%. The structure (FIG. 8A) was phased with molecular replacement using pdb code 1W2T as the search model and contains six monomers per asymmetric unit. The structure was refined with D-fructose bound in the active site with a final Rworking=0.179 and Rfree=0.208.

The structure of T. maritima invertase grown in the presence of D-fructose contains electron density in the active site that corresponds to a D-fructose-like molecule. A structure could not be definitively assigned to the density, but since the wild-type enzyme was crystalized with a demonstrated substrate, the density likely represents a mixture of products along the reaction path. D-fructose in FIG. 8B is modeled based on the orientation of raffinose (Alberto et al, 2006) and fits well into the electron density maps. Insight can be gained from this preliminary structure: cyclic products model better than linear ones and density corresponding to the 2′ hydroxyl is not evident in the maps. These results suggest the predicted chemistry (elimination of the 2′-hydroxyl, FIG. 4) is consistent with the initial structure.

### Example 2

### Development and Screening of Mutant Invertase Libraries

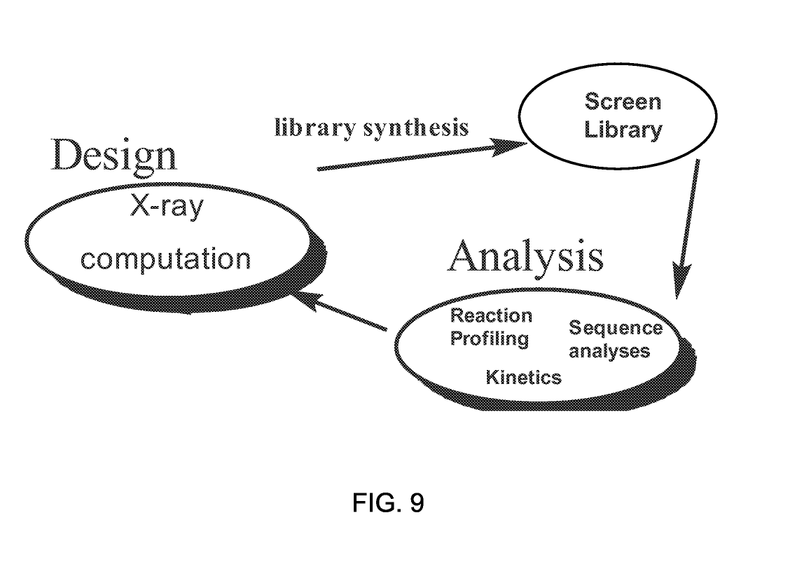

In this example saturation mutagenesis libraries were produced in invertase. The mutants were screened by HPLC to detect produced HMF, allowing identification of catalytically gifted mutants for additional profiling and study, as illustrated by the general process illustrated in FIG. 9. Using these methods, a catalyst was developed that approximately doubles the initial velocity of HMF production by wild-type invertase, while maintaining thermostability.

Engineering Invertase

As presented in Example 1 above, the wild type invertase is capable of the desired activity of producing HMF from fructose, and it is thermostable, so producing mutant invertase variants and selecting for improved variants was the goal of the present example. A combination of mutagenesis, X-ray crystallography, reaction profiling and kinetics were used for producing an analysis of invertase variants. In preliminary results a plausible kinetic bottleneck was identified after the first dehydration, and point to point contacts between bound substrate and the protein were targeted with saturation mutagenesis.

Library Design

The library design approach of this example used iterative saturation mutagenesis (ISM) (Reetz, 2007). With the advantage of high resolution structures initial choices were focused based on geometrical and chemical considerations. Candidates for the first round of mutagenesis were based on visual inspection of the X-ray crystallographic data and identification of the first shell of amino acid residues directly interacting with the bound substrate. (FIGS. 6, 7, 8B). Based on the structure, the first round of ISM employs generation of NNK degenerate libraries (all 20 amino acids) at each of the positions shown in FIG. 7. The D138 position was selected for the first round of mutagenesis. For each library approximately 100 colonies are screened for 95% coverage.

Library creation and screening, general method.

A modified version of the Stratagene QuikChange mutagenesis method was used that was adapted for library creation. (see, Steffens, 2007 and Sullivan 2013, which are incorporated by reference herein) Briefly, primers were synthesized containing NNK (and complementary NNM) degeneracies at the desired position. (N=A,T,C,G; K=G,T; M=C,A) They had overlapping regions of 25 bp, (Zheng, 2004) but were not entirely complementary, such that the resulting PCR products may serve as templates for subsequent cycles. This had the advantage of allowing lower levels of template and thus minimizing background levels of template found in the final library (Sullivan 2013). After polymerase reaction, the template was digested with Dpnl, and the library was transformed by electroporation into E. coli NEB5alpha. Pooled plasmids were sequenced at the University of Florida sequencing core to confirm the library quality (Sullivan, 2013).

If sequencing reveals codon bias that would have the effect of not properly sampling all amino acids, degenerate primers can be used that were hand enriched in sequences that were identified as depleted. This process results in the creation of diverse saturation mutagenesis libraries targeting loci selected through structural and mechanistic studies and identifies that library members adequately represent NNK degeneracy.

The plasmid library was transformed into E. coli BL21(DE3) and plated on LB/kanamycin agar media for screening. One hundred of the resulting colonies were selected from the plate and binned into groups of 20. The original plates were retained for eventual recovery of the plasmid identified in the screening process. Each group of 20 picked colonies were grown together in LB/kanamycin broth and then induced with IPTG to produce the N-terminal His tag protein sub-library. The cells were lysed by sonication and the debris removed by centrifugation. The supernate was applied to a nickel IMAC column and the sub-library of mutant invertases was eluted with an imidazole step gradient, typically up to 100 mM. Purified proteins were dialyzed against 10 mM phosphate buffer, pH 6, and concentrated to >1.0 mg/mL. Each of the 5 pools was then used to produce HMF from fructose using 1.0 mg/mL protein and 10 mM fructose. HPLC (C18, reverse phase, 97% water/3% acetonitrile, 1.0 mL/min was used to detect (284 nm UV) and quantify the amount of HMF produced relative to the wild-type invertase at the same total protein concentration. Libraries containing activity were then further split into 4 groups of 5 (derived from the group of 20 of the original 100 colonies selected) and assayed in the same way as described above, active sub-library was identified and so on until a single plasmid is identified that codes for a mutant invertase with enhanced catalytic properties.

Library Screening & Results

D138X

An invertase mutant library for amino acid D138 was created, with mutants having various D138X mutations (with X representing any other amino acid). The D138X invertase mutant libraries were created in the pET28 expression vector and screened using the above-described split and pool method, as described more specifically below.

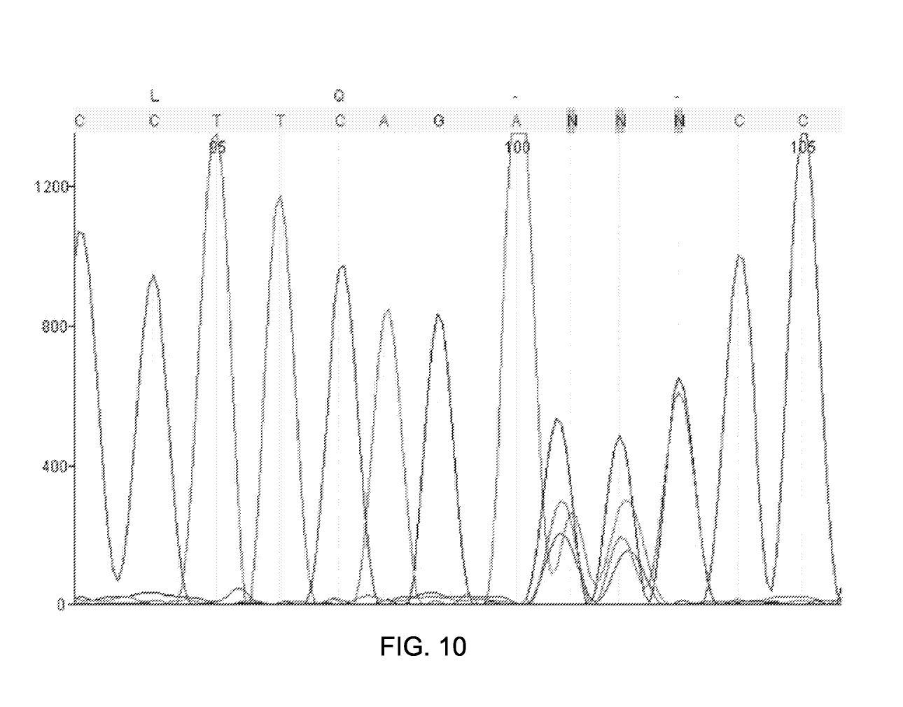

A sample of 100 colonies was found sufficient to ensure complete amino acid coverage at 90% confidence level. Sequence analysis of the D138X mutagenesis library illustrated by the electropherogram of FIG. 10 shows good coverage of NNK codon space.

Utilizing the above mentioned pool and split method the library was screening to yield identification of a single active member which was sequenced and found to be D138A. The pure mutant protein was then expressed and activity characterized by HPLC assay. The graph in FIG. 5 shows the kinetic characterization of both the wild type invertase (diamonds, middle) the D138A mutant (squares, top), illustrating the superior activity of the D138 mutant in producing HMF relative to the wild type invertase and absence of enzyme (triangles, bottom).

### Example 3

### Additional Engineering of Mutants with Enhanced HMF Production

After screening, candidates with enhanced HMF producing activity are then characterized and compared via the work described below for kinetic analyses and reaction profiling. Exceptional enzyme candidates are crystallized for structural analyses. These data are used to select the mutants from the first round that will be subjected to the second round of mutagenesis. For example, if positions 74, 138, and 240 are respectively found to yield mutants that are increasingly enhanced over wild type, double and triple mutants will be prepared. This approach assumes the contributions of each mutant are additive.

An alternate approach is to take the best of the three hypothetical candidates and use it for the second round. For example, if a position 240 mutant is the best catalyst, this mutant is then carried forward and a second round of ISM is performed on the remaining positions to identify the next candidate, which is a double mutant relative to the starting wild-type.

Finally, the library design process of the present example utilizes computational tools. Using the AMBER12 suite of programs MD studies are performed on experimentally determined structures to profile the dynamics of the protein. It has been observed that thermostability can be engineered by targeting particularly mobile spots, thus the example utilizes MD trajectories and B-factors from the experimental work. (Reetz, 2006) This becomes useful if a mutant series has favorable catalytic properties but experiences degradation of thermal stability.

### Example 4

### Conversion of Glucose/Xylose to HMF

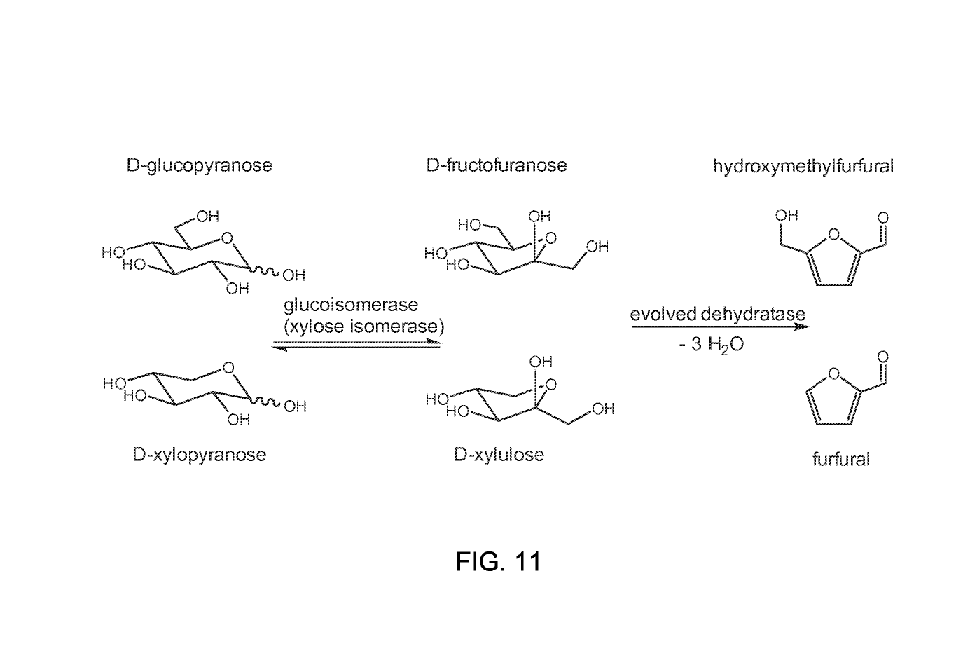

This example represents a comprehensive plan to produce an optimized enzyme for HMF and/or furfural production. This example uses the enzyme glucose isomerase in conjunction with an evolved catalyst (e.g., the engineered invertase described in Example 2) to produce HMF from glucose in a two step-enzymatic route, something that has never been accomplished before. (FIG. 11) Huang has employed glucose isomerase in a chemo-enzymatic route relying on acid catalyzed dehydration. (Huang, 2010) It is noteworthy that the Huang work and others have noted the stability of immobilized glucose isomerase towards organic solvents and ionic liquids. (Stahlberg, 2012) Glucose isomerase is well characterized, having been used industrially for over 50 years (Boshale, 1996; DiCosimo, 2013) Importantly, if the new catalysts of the present disclosure are capable of recognizing xylulose, glucose isomerase can also be used to produce this five carbon ketose from xylose (the preferred reaction of glucose isomerase). A mixture of xylose and glucose is a model for the primary carbohydrate components in lignocellulosic feedstocks, so accomplishing conversion of this mixture to furans is useful.

This example also tests glucose isomerase activity in the presence of high HMF. The first experiments survey glucose isomerase variants from different species and in immobilized and soluble forms for HMF tolerance, as evidenced by measurement of initial velocity of the enzyme towards the isomerization reaction after pre-incubating it with up to 1M HMF or furfural. Reaction kinetics are monitored by 1H-NMR. The impact of HMF concentration, temperature, and pH is determined from the results of these measurements.

Methods and compositions for one pot conversion of glucose or xylose/glucose mixtures into HMF and or HMF/furfural is described. Based upon the conditions identified above, reactions are conducted for conversion of glucose directly to HMF and conversion of mixtures of xylose and glucose to the HMF and furfural products. It is possible that at high turnovers there could be catalyst fouling due for example to covalent aldehyde/lysine imine formation. One approach to solving this is to reduce the reaction mixture in situ with NaCNBH3, identify the positions with MS-MS of tryptic digests, and possibly mutate them to Arg (for example) which would maintain charge but be less likely to react. This example provides one pot conversion of the aldose into HMF by strictly enzymatic methods. The target for the first generation system will be 50% conversion at gram scale.

### REFERENCES

Alberto, F.; Bignon, C.; Sulzenbacher, G.; Henrissat, B. and Czjzek, M. (2004) The three-dimensional crystal structure of invertase (b-fructosidase) from Thermotoga maritima reveals a bimodular arrangement and an evolutionary relationship between retaining and inverting glycosidases.” J. Biol. Chem. 279, 18903-18910.
Alberto, F.; Jordi, E.; Henrissat, B.; Czjzek, M. (2006). Crystal structure of inactivated Thermotoga maritima invertase in complex with the trisaccharide substrate raffinose. Biochem J, 395, 457-462.
Binder, J. B.; Raines, R. T. (2009) “Simple Chemical Transformation of Lignocellulosic Biomass into Furans for Fuels and Chemicals.” J. Am. Chem. Soc. 131, 1979-1985.
Bhosale, S. H.; Rao, M. B.; Deshpande, V. V. (1996) “Molecular and Industrial Aspects of Glucose Isomerase” Microbiol. Rev. 60, 280-300.
Burke, E. and Horenstein, N. A. (2004) “Enzymatic Synthesis of [1-¹⁴C-N-acetyl, P¹⁸O₂] Cytidine Monophosphate Neuraminic Acid” J. Label. Compd. Radiopharm. 47, 1007-1017.
Cagmat, E. B.; Szczepanski, J.; Pearson, W. L.; Powell, D. H.; Eyler, J. R.; Polfer, N. C.; (2010) “Vibrational signatures of metal-chelated monosaccharide epimers: gas-phase infrared spectroscopy of Rb⁺-tagged glucuronic and iduronic acid” Phys. Chem. Chem. Phys., 12, 3474-3479.
Chen, M. M. Y.; Snow, C. D.; Vizcarra, C. L.; Mayo, S. L.; Arnold, F. H. (2012) “Comparison of random mutagenesis and semi-rational designed libraries for improved cytochrome P450 BM3-catalyzed hydroxylation of small alkanes” Prot. Eng., Design & Selection, 25 171-178.
Capobianco, M.; Mezzina, E.; Savoia, D.; Tagliavini, E.; Trombini, C.; Umani-Ronchi, A. (1986)
Prostanoids from D-glucose. Palladium-catalyzed alkylation of 1,2-O-isopropylidene-3-deoxy-5-acetoxy-α-D-erythro-pent-5-en-furanose” Tett. Lett. 27, 1387-1390.
Chica, R. A.; Doucet, N.; Pelletier, J. N. (2005) “Semi-rational approaches to engineering enzyme activity: combining the benefits of directed evolution and rational design” Curr. Opin. Biotechnol., 16, 378-384.
Copley, S. D. (2003) “Enzymes with extra talents: moonlighting functions and catalytic promiscuity”, Curr. Opin. Chem. Biol. 7, 265-272.
Deuschle, U.; Kammerer, W.; Gentz, R.; Bujard, H. (1986) “Promoters of Escherichia coli: a hierarchy of in vivo strength indicates alternate structures” EMBO J. 5, 2987-2994.
DiCosimo, R.; McAuliffe, J.; Poulose, A. J.; Bohlmann, G. (2013) “Industrial use of immobilized enzymes” Chem. Soc. Rev., Advance Article DOI: 10.1039/C3CS35506C
D'Souza, F. W.; Ayers, J. D.; McCarren, P. R.; Lowary, T. L. (2000)“Arabinofuranosyl Oligosaccharides from Mycobacteria: Synthesis and Effect of Glycosylation on Ring Conformation and Hydroxymethyl Group Rotamer Populations” J. Am. Chem. Soc., 122, 1251-1260.
Ferenci, T.; Kornberg, H. L. (1971) “Pathway Of Fructose Utilization By Escherichia coli” Feb. Lett. 13, 127-130.
Gandini, A. (2010) “Furans as offspring of sugars and polysaccharides and progenitors of a family of remarkable polymers: a review of recent progress” Polym. Chem., 1, 245-251.
Garcia, J. G.; Voll, R. J.; Younathan, E. S. (1991) “Stereoselective Carbohydrate Synthesis Via Palladium Hydroxide Catalyzed Epoxide Hydrogenolysis” Tet. Lett. 32, 5273-5276.
Gentz, R.; Bujard, H. (1985) “Promoters Recognized by Escherichia coli RNA Polymerase Selected by Function: Highly Efficient Promoters from Bacteriophage T5” J. Bact. 164, 70-77.
Huang, R.; Qi, W.; Su R.; He, Z. (2010) “Integrating enzymatic and acid catalysis to convert glucose into 5-hydroxymethylfurfural” Chem. Comm. 46, 1115-1117.
Hutchison, C. A., Phillips, S., Edgell, M. H., Gillham, S., Jahnke, P., Smith, M. (1978) “Mutagenesis at a Specific Position in a DNA Sequence.” J. Biol. Chem. 253, 6551-6560.
Kirmizialtin, S.; Nguyen, V.; Johnson, K. A.; Elber, R. (2012) “How Conformational Dynamics of DNA Polymerase Select Correct Substrates: Experiments and Simulations”, Structure, 20, 618-627.
Liebl, W.; Brem, D.; Gotschlich, A. (1998) “Analysis of the gene for b-fructosidase (invertase, inulinase) of the hyperthermophilic bacterium Thermotoga maritima, and characterisation of the enzyme expressed in Escherichia coli” Appl Microbiol Biotechnol. 50, 55-64.
Miller, R. D.; McKean, D. R. (1982) “A facile preparation of methyl enol ethers from acetals and ketals using trimethylsilyl iodide” Tet. Lett. 23, 323-326.
Leonik, F. M.; Ghiviriga, I.; and Horenstein, N. A. (2010)“Synthesis of 3,5-diazabicyclo [5.1.0] octenes. A new platform to mimic glycosidase transition states.” Tetrahedron, 66, 5566-5572.
Lutz, S. (2010) “Beyond directed evolution-semi-rational protein engineering and design” Curr. Opin. Biotechnol., 21(6), 734-743.
Liu, Y.; Zheng, T. and Bruner, S. D. (2011) “Structural basis for phosphopantetheinyl carrier domain interactions in the terminal module of nonribosomal peptide synthetases. Chem Biol, 18, p. 1482-1488.
Lomas, J. S. (1981) “Primary and Secondary Kinetic Isotope Effects in the Acid-Catalyzed Dehydration of 1,l′-Diadamantylmethylcarbinol in Aqueous Acetic Acid” J. Org. Chem. 46, 412-415.
Lopes de Souza, R.; Yu, H.; Rataboul, F.; and Essayem, N. (2012) “5-Hydroxymethylfurfural (5-HMF) Production from Hexoses: Limits of Heterogeneous Catalysis in Hydrothermal Conditions and Potential of Concentrated Aqueous Organic Acids as Reactive Solvent System” Challenges, 3, 212-232.
Martinez, A.; Rodriguez, M. E.; York, S. W.; Preston, J. F.; Ingram, L. O. (2000) “Use of UV absorbance To monitor furans in dilute acid hydrolysates of biomass.” Biotechnol Prog. 16, 637-641.
Mino, W. K.; Gulyuz, K.; Wang, D.; Stedwell, C. N.; Polfer, N. C. (2011) “Gas-phase structure and dissociation chemistry of protonated tryptophan elucidated by infrared multiple-photon dissociation spectroscopy” J. Phys. Chem. Lett. 2, 299-304.
Morelle, W.; Michalski, J.-C. (2004) “Sequencing of oligosaccharides derivatized with benzylamine using electrospray ionization quadrupole time of flight-tandem mass spectrometry” Electrophoresis, 25, 2144-2155.
Navacchia, M. L.; Montevecchi, P. C. (2006) “Sulfanyl radical promoted C4′-C5′ bond scission of 5′-oxo-3′,4′-didehydro-2′,3′-dideoxynucleosides” Org. Biomol. Chem., 4, 3754-3756.
Northrop, D. B. (1981) “The Expression Of Isotope Effects On Enzyme-Catalyzed Reactions” Ann. Rev. Biochem. 50, 103-131.
Nowicki, M. W.; Tulloch, L. B.; Worrall, L.; McNae, I. W.; Hannaert, V.; Michels, P. A. M.; Fothergill-Gilmore, L. A.; Walkinshaw, M. D.; Turner, N. J. (2008) “Design, synthesis and trypanocidal activity of lead compounds based on inhibitors of parasite glycolysis” Bioorg. Med. Chem., 16, 5050-5061.
Otero, D. A.; Simpson, R. (1984) “2,5-Anhydro-D-Hexitols: Syntheses Of 2,5-Anhydro-D-Altritol and 2,5-Anhydro-D-Iditol” Carb. Res. 128, 79-86.
Polfer, N C; Oomens, J; (2009) “Vibrational spectroscopy of bare and solvated ionic complexes of biological relevance” Mass Spectrometry Reviews, 28, 468-494.
Quin, M. B.; Schmidt-Dannert, C. (2011) “Engineering of biocatalysts—from evolution to creation” ACS Catal. 1, 1017-1021.
Richter, F.; Leaver-Fay, A.; Khare, S. D.; Bjelic, S.; Baker, D. (2011) “De Novo Enzyme Design Using Rosetta3.” PLoS ONE 6(5), e19230. doi:10.1371/journal.pone.0019230
Roman-Leshkov, Y.; Chheda, J. N.; Dumesic, J. A. (2006) “Phase Modifiers Promote Efficient Production of Hydroxymethylfurfural from Fructose” Science 312, 1933-1937.
Romero, P. A; Arnold, F. (2009)“Exploring protein fitness landscapes by directed evolution” Nat Rev Mol Cell Biol. 10, 866-876.
Reetz, M. T.; Carballeira, J. D.; Vogel, A. (2006) “Iterative Saturation Mutagenesis on the Basis of B Factors as a Strategy for Increasing Protein Thermostability” Ang. Chem. Int. Ed. 45, 7745-7751.
Reetz, M. T.; Barballeira, J. D. (2007) “Iterative saturation mutagenesis (ISM) for rapid directed evolution of functional enzymes” Nature Protocols, 2, 891-903.
Rosatella, A. A.; Simeonov, S. P.; Frade, R. F. M.; Afonso, C. A. M. (2011) “5-Hydroxymethylfurfural (HMF) as a building block platform: Biological properties, synthesis and synthetic applications”. GreenChem. (2011), 13, 754-793.
Shi, B.; Dabbagh, H. A.; Davis, B. H. (2002) “Catalytic dehydration of alcohols. Kinetic isotope effect for the dehydration of t-butanol” Top. Catal. 18, 259-264.
Stahlberg, T.; Woodley, J. M.; Riisager, A. (2012) “Enzymatic isomerization of glucose and xylose in ionic liquids” Catal. Sci. Technol., 2, 291-295.
Stedwell, C. N.; Patrick, A. L.; Gulyuz, G.; Polfer, N. C. (2012) “Screening for phosphorylated peptides by infrared photodissociation spectroscopy” Anal. Chem., 84, 9907-9912.
Steffens, D. L.; Williams, J. G. K. (2007) “Efficient Site-Directed Saturation Mutagenesis Using Degenerate Oligonucleotides” J Biomol Tech. 18, 147-149.
Stevenson, B. J.; Yip, S. H.-C.; Ollis, D. L. (2013) “In Vitro Directed Evolution of Enzymes Expressed by E. coli in Microtiter Plates” Meth. Mol. Biol. 978, 237-249.
Sullivan, B.; Walton, A. Z.; Stewart, J. D. (2013, in press) “Library Construction and Evaluation for Site Saturation Mutagenesis” Enz. Microbial Tech. http://dx.doi.org/10.1016/j.enzmictec.2013.02.012
Tantillo, D. J.; Chen, J.; Houk, K. N. (1998) “Theozymes and Compuzymes: Theoretical Models for Biological Catalysis” Curr. Op. Chem. Biol. 2, 743-750.
Turanli-Yildiz, B.; Alkim C.; Cakar, Z. P. (2012) “Protein Engineering Methods and Applications” Chapter 2, pp 33-58, in “Protein Engineering” Edited by Pravin Kaumaya, InTech. DOI: 10.5772/1286
Vocadlo, D. J.; Davies, G. J. (2008) “Mechanistic insights into glycosidase chemistry” Curr. Opin. Chem. Biol., 12, 539-555.
Westheimer, F. H. (1961) “The Magnitude of the Primary Kinetic Isotope Effect for Compounds of Hydrogen and Deuterium” Chem. Rev. 61, 265-73
Widboom, P. F.; Fielding, E. N.; Liu, Y. and Bruner, S. D. (2007) “Structural basis for cofactor-independent dioxygenation in vancomycin biosynthesis.” Nature, 447, p. 342-345.
Zheng, L., Baumann, U. and Reymond, J.-L. (2004) An Efficient One-Step Site-Directed and Site Saturation Mutagenesis Protocol. Nucl. Acids Res., 32, e115.

Protein sequence of β-fructofuranosidase
from T. martitima
(SEQ ID NO: 1)
MFKPNYHFFPITGWMNDPNGLIFWKGKYHMFYQYNPRKPEWGNICWG
HAVSDDLVHWRHLPVALYPDDETHGVFSGSAVEKDGKMFLVYTYYRD
PTHNKGEKETQCVAMSENGLDFVKYDGNPVISKPPEEGTHAFRDPKV
NRSNGEWRMVLGSGKDEKIGRVLLYTSDDLFHWKYEGVIFEDETTKE
IECPDLVRIGEKDILIYSITSTNSVLFSMGELKEGKLNVEKRGLLDH
GTDFYAAQTFFGTDRVVVIGWLQSWLRTGLYPTKREGWNGVMSLPRE
LYVENNELKVKPVDELLALRKRKVFETAKSGTFLLDVKENSYEIVCE
FSGEIELRMGNESEEVVITKSRDELIVDTTRSGVSGGEVRKSTVEDE
ATNRIRAFLDSCSVEFFFNDSIAFSFRIHPENVYNILSVKSNQVKLE
VFELENIWL
Nucleotide sequence of β-fructofuranosidase
from T. martitima (gi|15642775:c1430112-1428814
Thermotoga maritima MSB8 chromosome, complete
genome)
(SEQ ID NO: 2)
ATGTTCAAGCCGAATTATCACTTTTTCCCGATAACAGGCTGGATGAA
CGATCCGAACGGTTTGATCTTCTGGAAGGGAAAATATCATATGTTCT
ATCAGTATAATCCCAGAAAACCTGAGTGGGGAAACATCTGCTGGGGC
CACGCGGTGAGCGACGATCTCGTTCACTGGAGACACCTTCCCGTTGC
TCTATATCCCGACGATGAAACACACGGAGTGTTCTCTGGAAGCGCTG
TCGAGAAAGATGGGAAAATGTTTCTCGTGTACACCTACTACCGCGAT
CCGACACACAACAAAGGAGAAAAAGAAACCCAGTGTGTGGCTATGAG
TGAAAACGGATTGGATTTCGTAAAGTACGATGGAAACCCGGTCATAT
CTAAACCCCCAGAGGAAGGGACGCACGCCTTCAGAGACCCGAAGGTG
AACAGAAGCAACGGTGAGTGGCGAATGGTACTGGGATCTGGTAAAGA
TGAGAAGATTGGAAGAGTGCTTCTCTATACCTCAGATGACCTTTTTC
ACTGGAAGTACGAGGGTGTGATCTTCGAAGATGAAACCACAAAAGAA
ATAGAGTGTCCCGATCTTGTGAGAATTGGAGAGAAAGATATCCTCAT
ATACTCGATAACGAGTACAAACAGCGTTCTGTTTTCCATGGGAGAGT
TAAAGGAAGGAAAACTGAATGTCGAAAAGCGGGGGCTTCTCGATCAC
GGAACGGATTTCTACGCTGCTCAAACTTTCTTTGGAACAGACAGAGT
TGTAGTTATCGGATGGCTTCAAAGCTGGTTGAGAACAGGGCTTTACC
CGACAAAACGAGAAGGATGGAACGGTGTCATGAGTCTTCCTAGGGAG
CTGTATGTAGAAAACAACGAGTTGAAGGTGAAACCGGTGGATGAACT
CTTGGCTCTCAGAAAGAGAAAGGTTTTCGAAACTGCAAAGTCCGGAA
CATTTCTGCTGGATGTCAAGGAAAACAGTTATGAAATTGTGTGTGAA
TTCAGCGGAGAAATCGAACTTCGAATGGGAAATGAATCTGAAGAAGT
GGTGATAACGAAGAGTCGAGACGAATTAATCGTGGATACAACGAGAT
CTGGTGTTTCAGGTGGAGAAGTTAGAAAGTCGACAGTCGAAGATGAA
GCTACAAATAGAATACGAGCTTTCTTGGATTCGTGTTCTGTAGAATT
TTTCTTCAACGACTCCATAGCTTTTTCCTTTAGGATCCATCCAGAGA
ACGTTTACAACATTCTTTCTGTCAAATCGAACCAAGTGAAACTCGAA
GTCTTTGAACTCGAGAACATATGGTTGTGA
Codon Optimized Nucleotide sequence of
β-fructofuranosidase from T. martitima
Seq2 (synthesized and used to make the same
invertase protein (SEQ ID NO: 1, but has a
different DNA sequence to codon optimize for
e. coli expression)
(SEQ ID NO: 3)
ATGTTCAAGCCGAATTATCACTTTTTCCCGATAACCGGTTGGATGAA
CGATCCGAACGGTTTGATCTTCTGGAAGGGAAAATATCACATGTTCT
ATCAGTATAATCCCAGAAAACCTGAGTGGGGAAACATCTGCTGGGGC
CACGCGGTGAGCGACGATCTCGTTCACTGGAGACACCTTCCCGTTGC
TCTATATCCCGACGATGAAACACACGGAGTGTTCTCTGGAAGCGCTG
TCGAGAAAGATGGGAAAATGTTTCTCGTGTACACCTACTACCGCGAT
CCGACACACAACAAAGGAGAAAAAGAAACCCAGTGTGTGGCTATGAG
TGAAAACGGATTGGATTTCGTAAAGTACGATGGAAACCCGGTCATAT
CTAAACCCCCAGAGGAAGGGACGCACGCCTTCAGAGACCCGAAGGTG
AACAGAAGCAACGGTGAGTGGCGAATGGTACTGGGATCTGGTAAAGA
TGAGAAGATTGGAAGAGTGCTTCTCTATACCTCAGATGACCTTTTTC
ACTGGAAGTACGAGGGTGTGATCTTCGAAGATGAAACCACAAAAGAA
ATAGAGTGTCCCGATCTTGTGAGAATTGGAGAGAAAGATATCCTCAT
ATACTCGATAACGAGTACAAACAGCGTTCTGTTTTCCATGGGAGAGT
TAAAGGAAGGAAAACTGAATGTCGAAAAGCGGGGGCTTCTCGATCAC
GGAACGGATTTCTACGCTGCTCAAACTTTCTTTGGTACCGACAGAGT
TGTAGTTATCGGATGGCTTCAAAGCTGGTTGAGAACAGGGCTTTACC
CGACAAAACGAGAAGGATGGAACGGTGTCATGAGTCTTCCTAGGGAG
CTGTATGTAGAAAACAACGAGTTGAAGGTGAAACCTGTGGATGAACT
CTTGGCTCTCAGAAAGAGAAAGGTTTTCGAAACTGCAAAGTCCGGAA
CATTTCTGCTGGATGTCAAGGAAAACAGTTATGAAATTGTGTGTGAA
TTCAGCGGAGAAATCGAACTTCGAATGGGAAATGAATCTGAAGAAGT
GGTGATAACGAAGAGTCGAGACGAATTAATCGTGGATACAACGAGAT
CTGGTGTTTCAGGTGGAGAAGTTAGAAAGTCGACAGTCGAAGATGAA
GCTACAAATAGAATACGAGCTTTCTTGGATTCGTGTTCTGTAGAATT
TTTCTTCAACGACTCCATAGCTTTTTCCTTTAGGATCCATCCAGAGA
ACGTTTACAACATTCTTTCTGTCAAATCGAACCAAGTGAAACTCGAA
GTCTTTGAACTCGAGAATATTTGGTTGTGA

1. A method of producing hydroxymethylfurfural (HMF), the method comprising:
providing a carbohydrate composition comprising fructose, one or more carbohydrates that can be converted to fructose, or a combination thereof;
exposing the carbohydrate composition to an enzyme composition comprising at least one isolated enzyme capable of catalyzing dehydration of fructose to HMF; and
recovering the HMF produced by reaction of the fructose and the enzyme.

2. The method of claim 1, wherein the enzyme is a glycosidase.

3. The method of claim 2, wherein the glycosidase is an invertase.

4. The method of claim 3, wherein the invertase is β-fructofuranosidase from T. maritima or a functional variant thereof.

5. The method of claim 1, wherein the carbohydrate composition comprises glucose and the method further comprises exposing the carbohydrate composition to an isomerase enzyme capable of converting glucose to fructose.

6. The method of claim 5, wherein the isomerase enzyme is xylose isomerase.

7. (canceled)

8. The method of claim 6, wherein the enzyme capable of catalyzing dehydration of fructose to HMF is an invertase and wherein the xylose isomerase is coupled to the invertase.

9. The method of claim 4, wherein the invertase is a functional variant of β-fructofuranosidase having at least one improvement over the corresponding native protein, wherein the improvement comprises one or more improvements selected from the group consisting of: increased activity, increased stability, increased rate of catalysis, and increased efficiency.

10. The method of claim 4, wherein the β-fructofuranosidase has the peptide sequence of SEQ ID NO: 1, or a variant of SEQ ID NO: 1 having the mutation D138A.

11. (canceled)

12. An enzyme capable of catalyzing dehydration of fructose to produce hydroxymethylfurfural (HMF) comprising:
a functional variant of a native invertase protein capable of catalyzing dehydration of fructose to HMF, the variant comprising at least one engineered mutation relative to the native invertase, wherein the variant has a least one improvement with respect to the native invertase, the improvement comprising one or more improvements selected from the group consisting of: increased activity in catalyzing the dehydration of fructose to HMF, increased stability, increased rate of catalysis of fructose to HMF, and increased efficiency.

13. The enzyme of claim 12, wherein the native invertase is β-fructofuranosidase from T. maritima.

14. The enzyme of claim 12, wherein the native invertase is β-fructofuranosidase having the peptide sequence of SEQ ID NO: 1.

15. The enzyme of claim 14, wherein the at least one engineered mutation in the variant of native β-fructofuranosidase comprises a mutation involving one or more of the following amino acid residues of SEQ ID NO: 1: N16, D17, Q33, W41, F74, S75, R137, D138, E190, Y240, A241, and W260.

16. The enzyme of claim 15, wherein the at least one engineered mutation comprises D138A.

17. The enzyme of claim 12, wherein the enzyme has increased activity as compared to the native invertase and is at least as stable as the native invertase.

18. The enzyme of claim 12 further comprising a xylose isomerase enzyme coupled to the functional variant of the native invertase protein, wherein the xylose isomerase enzyme is capable of catalyzing conversion of glucose to fructose, whereby the coupled enzymes form a fusion protein capable of catalyzing production of HMF from glucose.

19. (canceled)

20. The enzyme of claim 12, wherein the xylose isomerase is further capable of catalyzing the conversion of xylose into furfural.

21. A method of making a novel invertase capable of catalyzing dehydration of fructose to hydroxymethylfurfural (HMF), the method comprising:
selecting a native invertase protein capable of catalyzing dehydration of fructose to HMF;
creating a library of variants of the native invertase protein, each variant having at least one mutation with respect to the native protein;
selecting functional variants from the library; and
testing selected functional variants for improvements with respect to the native protein, the improvement comprising one or more improvements selected from the group consisting of: increased activity in catalyzing the dehydration of fructose to HMF, increased stability, increased rate of catalysis of fructose to HMF, and increased efficiency.

22-31. (canceled)

32. The method of claim 21, wherein the native invertase is β-fructofuranosidase from T. maritima.

33. The method of claim 21, wherein the native invertase is β-fructofuranosidase comprising SEQ ID NO: 1 and wherein the library of variants comprises at least one variant comprising a mutation involving one or more of the following amino acid residues of SEQ ID NO: 1: N16, D17, Q33, W41, F74, S75, R137, D138, E190, Y240, A241, and W260.


SEQUENCE LISTING (3 sequences)

SEQ ID NO: 1 - protein, 432 aa, Thermotoga martitima
MFKPNYHFFPITGWMNDPNGLIFWKGKYHMFYQYNPRKPEWGNICWGHAVSDDLVHWRHLPVALYPDDETHGVFSGSAVEKDGKMFLVYTYYRDPTHNKGEKETQCVAMSENGLDFVKYDGNPVISKPPEEGTHAFRDPKVNRSNGEWRMVLGSGKDEKIGRVLLYTSDDLFHWKYEGVIFEDETTKEIECPDLVRIGEKDILIYSITSTNSVLFSMGELKEGKLNVEKRGLLDHGTDFYAAQTFFGTDRVVVIGWLQSWLRTGLYPTKREGWNGVMSLPRELYVENNELKVKPVDELLALRKRKVFETAKSGTFLLDVKENSYEIVCEFSGEIELRMGNESEEVVITKSRDELIVDTTRSGVSGGEVRKSTVEDEATNRIRAFLDSCSVEFFFNDSIAFSFRIHPENVYNILSVKSNQVKLEVFELENIWL

SEQ ID NO: 2 - DNA, 1299 bp, Thermotoga martitima
atgttcaagccgaattatcactttttcccgataacaggctggatgaacgatccgaacggtttgatcttctggaagggaaaatatcatatgttctatcagtataatcccagaaaacctgagtggggaaacatctgctggggccacgcggtgagcgacgatctcgttcactggagacaccttcccgttgctctatatcccgacgatgaaacacacggagtgttctctggaagcgctgtcgagaaagatgggaaaatgtttctcgtgtacacctactaccgcgatccgacacacaacaaaggagaaaaagaaacccagtgtgtggctatgagtgaaaacggattggatttcgtaaagtacgatggaaacccggtcatatctaaacccccagaggaagggacgcacgccttcagagacccgaaggtgaacagaagcaacggtgagtggcgaatggtactgggatctggtaaagatgagaagattggaagagtgcttctctatacctcagatgacctttttcactggaagtacgagggtgtgatcttcgaagatgaaaccacaaaagaaatagagtgtcccgatcttgtgagaattggagagaaagatatcctcatatactcgataacgagtacaaacagcgttctgttttccatgggagagttaaaggaaggaaaactgaatgtcgaaaagcgggggcttctcgatcacggaacggatttctacgctgctcaaactttctttggaacagacagagttgtagttatcggatggcttcaaagctggttgagaacagggctttacccgacaaaacgagaaggatggaacggtgtcatgagtcttcctagggagctgtatgtagaaaacaacgagttgaaggtgaaaccggtggatgaactcttggctctcagaaagagaaaggttttcgaaactgcaaagtccggaacatttctgctggatgtcaaggaaaacagttatgaaattgtgtgtgaattcagcggagaaatcgaacttcgaatgggaaatgaatctgaagaagtggtgataacgaagagtcgagacgaattaatcgtggatacaacgagatctggtgtttcaggtggagaagttagaaagtcgacagtcgaagatgaagctacaaatagaatacgagctttcttggattcgtgttctgtagaatttttcttcaacgactccatagctttttcctttaggatccatccagagaacgtttacaacattctttctgtcaaatcgaaccaagtgaaactcgaagtctttgaactcgagaacatatggttgtga

SEQ ID NO: 3 - DNA, 1299 bp, Artificial Sequence
  chemically synthesized codon optimized nucleotide sequence of Beta-fructofuranosidase
atgttcaagccgaattatcactttttcccgataaccggttggatgaacgatccgaacggtttgatcttctggaagggaaaatatcacatgttctatcagtataatcccagaaaacctgagtggggaaacatctgctggggccacgcggtgagcgacgatctcgttcactggagacaccttcccgttgctctatatcccgacgatgaaacacacggagtgttctctggaagcgctgtcgagaaagatgggaaaatgtttctcgtgtacacctactaccgcgatccgacacacaacaaaggagaaaaagaaacccagtgtgtggctatgagtgaaaacggattggatttcgtaaagtacgatggaaacccggtcatatctaaacccccagaggaagggacgcacgccttcagagacccgaaggtgaacagaagcaacggtgagtggcgaatggtactgggatctggtaaagatgagaagattggaagagtgcttctctatacctcagatgacctttttcactggaagtacgagggtgtgatcttcgaagatgaaaccacaaaagaaatagagtgtcccgatcttgtgagaattggagagaaagatatcctcatatactcgataacgagtacaaacagcgttctgttttccatgggagagttaaaggaaggaaaactgaatgtcgaaaagcgggggcttctcgatcacggaacggatttctacgctgctcaaactttctttggtaccgacagagttgtagttatcggatggcttcaaagctggttgagaacagggctttacccgacaaaacgagaaggatggaacggtgtcatgagtcttcctagggagctgtatgtagaaaacaacgagttgaaggtgaaacctgtggatgaactcttggctctcagaaagagaaaggttttcgaaactgcaaagtccggaacatttctgctggatgtcaaggaaaacagttatgaaattgtgtgtgaattcagcggagaaatcgaacttcgaatgggaaatgaatctgaagaagtggtgataacgaagagtcgagacgaattaatcgtggatacaacgagatctggtgtttcaggtggagaagttagaaagtcgacagtcgaagatgaagctacaaatagaatacgagctttcttggattcgtgttctgtagaatttttcttcaacgactccatagctttttcctttaggatccatccagagaacgtttacaacattctttctgtcaaatcgaaccaagtgaaactcgaagtctttgaactcgagaatatttggttgtga